(19) United States
(12) Patent Application Publication
(10) Pub. No.: US 2010/0104551 A1
(43) Pub. Date: Apr. 29, 2010

(54) METHOD FOR PROLONGING ACTIVITY OF AUTODEGRADABLE ENZYMES AND COMPOSITIONS THEREOF

(75) Inventors: Dharmendra M. Jani, Fairport, NY (US); Kai Kwok, Rochester, NY (US); Gregory L. McIntire, Rochester, NY (US); Bruce A. Pfeffer, Fairport, NY (US); Afshin Shafiee, Rochester, NY (US); Ruiwen Shi, Cupertino, CA (US); Srini Venkatesh, Pittsford, NY (US); Hongna Wang, Fairport, NY (US); Yan Huang, Webster, NY (US); Stephen R. Davio, Fairport, NY (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Talecris Biotherapeutics, Inc., Research Triangle Park, NC (US)

(21) Appl. No.: 12/603,988

(22) Filed: Oct. 22, 2009

Related U.S. Application Data
(62) Division of application No. 11/601,389, filed on Nov. 17, 2006, now abandoned.
(60) Provisional application No. 60/749,806, filed on Dec. 13, 2005.

(51) Int. Cl. A61K 38/48; A61P 27/02

(52) U.S. Cl. 424/94.64

(57) ABSTRACT

A composition of a long-acting enzyme comprises the enzyme in a formulation comprising a buffer and an additive selected from the group consisting of tranexamic acid, ε-aminocaproic acid, and analogs of L-lysine other than tranexamic acid and ε-aminocaproic acid, combinations thereof, and mixtures thereof. The composition can further comprise another additive selected from the group consisting of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, gelatin, HSA, streptokinase, tPA, uPA, non-ionic surfactants, glycerin, D-sorbitol, combinations thereof, and mixtures thereof. A method for prolonging the activity of an autodegradable enzyme comprises storing the enzyme after manufacture at a low pH, and reconstituting the acidified enzyme before use with a solution containing at least one of such additives. The method is useful to provide enzyme for wide use, which otherwise would lose activity upon long storage. In one embodiment the method is applicable to provide enzyme for inducing controlled posterior vitreous detachment.

### BACKGROUND OF THE INVENTION

The present invention relates to a method for prolonging the activity of autodegradable enzymes and compositions thereof. In particular, the present invention relates to a method for prolonging the enzymatic activity of plasmin or its derivatives and compositions thereof. More particularly, the present invention relates to a method for obtaining extended in-vivo enzymatic activity of plasmin or derivatives thereof after storage and to a method for effecting posterior vitreous detachment using such plasmin or derivatives thereof.

Proteases (or proteolytic enzymes or peptidases) are enzymes that catalyze the degradation or breakdown of proteins and, thus, participate in many important physiologic processes. A protease or peptidase can be further classified as an endopeptidase (which cleave peptide bonds within a protein) or exopeptidase (which removes amino acids sequentially from either the N- or the C-terminus of a protein). An endopeptidase is also termed a “proteinase.” Plasmin, a serine proteinase, is the principal fribrinolytic enzyme in mammals, and has the important function of breaking down in-vivo blood clots. It derives from the inactive precursor plasminogen, which circulates in plasma at a concentration of about 1.5 μM. Circulating plasminogen is activated, for example in vivo, by plasminogen activators, such as tissue plasminogen activator (“tPA”) or urokinase, which cleave a single-chain plasminogen molecule at the Arg⁵⁶⁶-Val⁵⁶¹ peptide bond, producing active plasmin. Plasminogen is also activatable by the bacteria-derived enzyme streptokinase. Thus, thrombolytic drugs, such as those based on tPA, streptokinase, and urokinase-type plasminogen activator, have been developed for administering into patients suffering from various thrombotic disorders, including myocardial infarction, occlusive stroke, deep venous thrombosis, and peripheral arterial disease, to promote the in-vivo production of plasmin in order rapidly to enhance the degradation of blood clots. However, the administered tPA, streptokinase, or urokinase-type plasminogen activator still must encounter the circulating plasminogen in order to generate active plasmin, and the magnitude of the effectiveness of these thrombolytic drugs still depends on the inherent in-vivo level of plasminogen. Therefore, it has been thought that a higher benefit should be obtained if active plasmin is administered instead into these patients.

Plasmin also has been proposed for inducing controlled posterior vitreous detachment (“PVD”) to prevent, stop, or reduce the progression of retinal detachment. U.S. patent application Ser. No. 11/126,625 having the common assignee teaches that creation a PVD is thought to inhibit the progression of nonproliferative diabetic retinopathy. The references disclosed in that application are incorporated herein by reference.

The vitreous is a clear, proteinaceous mass which fills the posterior cavity of the eye between the lens and the retina. The vitreous is attached at its posterior face to the retina along the structure known as the internal limiting membrane. This site of attachment of the vitreous and the retina is termed the vitreoretinal junction and consists of a layer of basement membrane proximal to the retina and a layer of collagen fibrils proximal to the vitreous.

Degenerative changes in the vitreous are a precursor to posterior vitreous detachment (“PVD”). Degeneration of the vitreous is part of the normal aging process, but also may be induced by pathological conditions such as diabetes, Eales' disease and uveitis (see, e.g., “Retinal Detachment” at http://www.emedicine.com/emerg/topic504.html). Because the vitreous is attached to the retina, the receding vitreous can cause a retinal tear, with subsequent detachment of the retina.

Certain pathological conditions of the eye are accompanied by the formation of new (abnormal) vessels on the surface of the retina—namely proliferative diseases. With a naturally occurring PVD, traction is placed on new vessels causing rupture and bleeding. Proliferative retinal diseases thus are accompanied by both a high probability of retinal detachment as well as complications from bleeding resulting from the rupture of the newly formed blood vessels. Thus, it is beneficial to induce a controlled PVD before damage to the retina occurs because of uncontrolled detachment. Further, it is thought that attachments between the vitreous and the retina can serve as a scaffold for the growth of these new blood vessels through the retina and into the vitreous. Thus, creation of a PVD may avoid or inhibit such growth of blood vessels into the vitreous.

Verstraeten et al. (Arch. Opthalmol., Vol. 11, 849-854 (1993)) proposed the use of plasmin to produce a cleavage at the vitreoretinal interface. Plasmin hydrolyzes glycoproteins, including laminin and fibronectin, which are found at the vitreoretinal junction. Plasmin treatment was performed with or without subsequent vitrectomy on rabbit eyes. The authors noted that eyes treated with plasmin showed some areas of PVD, but only after vitrectomy was the vitreous substantially detached. The authors concluded that plasmin treatment may be useful as a biochemical adjunct to mechanical vitrectomy. However, plasmin rapidly autodegrades at or near physiological pH, at which it has the highest activity, and has not been available for therapeutic administration, as it cannot be stored at this pH. Therefore, U.S. Pat. No. 6,355,243, for example, teaches that isolated plasmin is stored at pH less than about 4 to avoid its autodegradation. However, when plasmin at such a low pH is administered into a patient whose physiological pH is about 7.4, undesirable effects may occur, such as precipitation due to the pH shift. In addition, in the vitreous, an interaction between plasmin and hyaluronic acid can also result in precipitation, rendering the enzyme less or completely inactive.

Therefore, there is a need to provide compositions comprising enzymes having activity at or near that at the time of its manufacture, after a prolonged storage, and methods for obtaining such enzymes. In addition, it is also desirable to provide a method for prolonging the activity of an enzyme after it has been administered into a patient. Moreover, it is also desirable to provide a method for stabilizing plasmin and derivatives thereof during storage, regaining their activity when they are ready for use, and prolonging their activity in vivo after administration into a patient, and compositions comprising such stabilized plasmin or its derivatives.

### SUMMARY OF THE INVENTION

In general, the present invention provides a composition comprising an active enzyme after prolonged storage and methods for making and using such a composition.

In one aspect, a composition of the present invention comprises an enzyme that has been preserved at a pH less than about 5 and is subsequently reconstituted in a formulation comprising a material selected from the group consisting of tranexamic acid (trans-4-(aminomethyl)cyclohexanecarboxylic acid) (sometimes abbreviated herein as “TXA”), ε-aminocaproic acid (sometimes abbreviated herein as “ε-ACA”), analogs of L-lysine other than tranexamic acid and ε-aminocaproic acid, combinations thereof, and mixtures thereof; wherein said enzyme is autodegradable at a pH greater than about 5.

In another aspect, the formulation further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), streptokinase, tPA, uPA, combinations thereof, and mixtures thereof; and Group 3 consists of non-ionic surfactants, glycerin, D-sorbitol, combinations thereof, and mixtures thereof.

In still another aspect, the formulation has a pH corresponding approximately to a pH at which said enzyme has the highest activity in a preselected reaction or use.

In still another aspect, the present invention provides a method for producing an active enzyme after prolonged storage, the method comprising: (a) storing said enzyme at a pH less than about 5; and (b) adding said enzyme to a formulation that comprises a material selected from the group consisting of tranexamic acid, ε-aminocaproic acid, analogs of L-lysine other than tranexamic acid and ε-aminocaproic acid, combinations thereof, and mixtures thereof; wherein said enzyme is autodegradable at pH greater than about 5.

In still another aspect, the formulation has a pH corresponding approximately to a pH at which said enzyme has the highest activity in a preselected reaction or use.

In still another aspect, the formulation used in the foregoing method further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), streptokinase, tPA, uPA, combinations thereof, and mixtures thereof; and Group 3 consists of non-ionic surfactants, glycerin, D-sorbitol, combinations thereof, and mixtures thereof; to produce a buffered enzyme composition substantially immediately before using the enzyme or carrying out the reaction.

In one embodiment, the non-ionic surfactants are selected from the group consisting of polysorbates, poloxamers, poloxamines, and mixtures thereof.

In yet another aspect, the enzyme is a proteolytic enzyme (or alternatively termed “protease,” or “peptidase,” or “proteinases”).

In a further aspect, the enzyme is selected from the group consisting of serine proteinases, cysteine proteinases, aspartyl proteinases, metalloproteinases (or alternatively termed “matrix metalloproteinases”), combinations thereof, and mixtures thereof. In one embodiment, the enzyme is tissue plasminogen activator (“tPA”), urokinase-type plasminogen activator (“uPA”), or streptokinase.

In still another aspect, the present invention provides a method for prolonging an activity of an enzyme at physiological pH, which enzyme is autodegradable at physiological pH, the method comprising: (a) providing said enzyme that has been preserved at a pH less than about 5; and (b) adding said enzyme to a formulation that has approximately physiological pH and comprises: (1) a material selected from the group consisting of tranexamic acid (trans-4-(aminomethyl)cyclohexanecarboxylic acid), ε-aminocaproic acid, analogs of L-lysine other than tranexamic acid and ε-aminocaproic acid, combinations thereof, or mixtures thereof; and (2) a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), streptokinase, tPA, uPA, combinations thereof, and mixtures thereof; and Group 3 consists of non-ionic surfactants, glycerin, D-sorbitol, combinations thereof, and mixtures thereof; to produce a buffered enzyme before administering said buffered enzyme into a patient, thereby prolonging the activity of said enzyme in said patient, wherein the post-administering enzyme activity in said patient is higher than the activity of enzyme in a formulation devoid of said buffer and said compound.

In yet another aspect, the present invention provides a method for preventing or reducing a precipitation of an enzyme administered into a vitreous of an eye, the method comprising: (a) providing the enzyme at a pH of less than about 5; (b) adding said enzyme to a formulation that comprises an additive selected from the group consisting of tranexamic acid, ε-aminocaproic acid, γ-aminobutyric acid, 5-aminovaleric acid, 7-aminoheptanoic acid, glycylglycine, triglycine, L-ornithine hydrochloride, N-α-acetyl-L-arginine, betaine, sarcosine, L-arginine, glycerin, D-sorbitol, gelatin, combinations thereof, and mixtures thereof; to produce a formulated enzyme substantially immediately before using said enzyme or carrying out the reaction; said enzyme is autodegradable at pH greater than about 5.

In a further aspect, the formulation has a pH in a range from about 6.5 to about 11.

In a further aspect, the present invention provides a method for inducing PVD in an eye, the method comprising: (a) providing plasmin or derivatives thereof that have been preserved at a pH less than about 5; and (b) adding said plasmin or derivatives thereof to a formulation having a pH in a range from about 6.5 to about 11; and comprising: (1) a material selected from the group consisting of tranexamic acid (trans-4-(aminomethyl)cyclohexanecarboxylic acid), ε-aminocaproic acid, analogs of L-lysine other than tranexamic acid and ε-aminocaproic acid, combinations thereof, or mixtures thereof; and (2) a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), combinations thereof, and mixtures thereof; and Group 3 consists of non-ionic surfactants, glycerin, D-sorbitol, combinations thereof, and mixtures thereof; to produce a buffered plasmin or derivatives thereof before administering said buffered plasmin or derivatives thereof into a posterior chamber of the eye, thereby inducing PVD in said eye.

Other features and advantages of the present invention will become apparent from the following detailed description and claims and the appended drawings.

### BRIEF DESCRIPTION OF THE DRAWINGS

FIG. 1 shows the pH dependence of the enzymatic activity of plasmin, as measured by the hydrolysis of peptidase substrate S-2251.

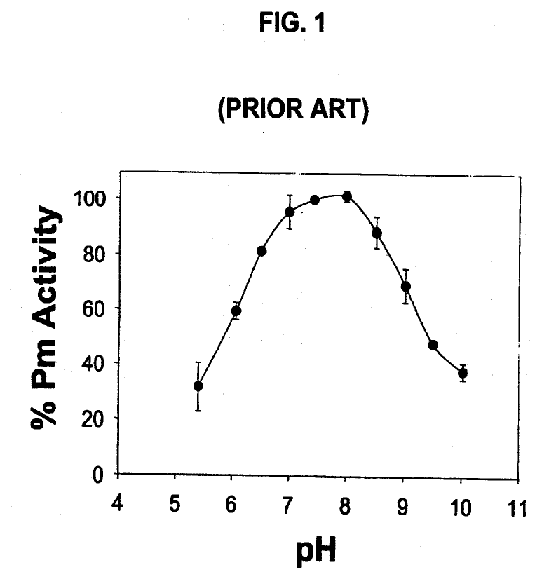

FIG. 2 shows plasmin activity in buffers containing 40 mM ε-aminocaproic acid or tranexamic acid.

FIG. 3 shows plasmin activity in buffers containing 40 mM ε-aminocaproic acid and another additive.

FIG. 4 shows plasmin activity in buffers containing 40 mM ε-aminocaproic acid and other additives, as indicated.

FIG. 5 shows plasmin activity in buffers containing 0.4 M γ-aminobutyric acid, 0.5 M L-ornithine hydrochloride, or 0.5 M glycylglycine.

FIG. 6 shows plasmin activity in buffers containing L-lysine hydrochloride or tranexamic acid.

FIG. 7 shows the effect of ε-aminocaproic acid on plasmin activity in pig vitreous at 37° C.

FIG. 8 shows the effect of diglycine on plasmin activity in pig vitreous at 37° C.

FIG. 9 shows the effects of 5-aminovaleric acid on plasmin activity in pig vitreous at 37° C.

FIG. 10 shows the effects of betaine and streptokinase on plasmin activity in pig vitreous at 37° C.

FIG. 11 shows the effect of TXA on plasmin activity in pig vitreous at 37° C.

FIG. 12 shows the effect of ε-aminocaproic acid on plasmin activity in pig vitreous at 37° C.

FIG. 13 shows the effects of various concentrations of TXA in combination with Tween 80® on plasmin activity in pig vitreous at 37° C.

FIG. 14 shows the effect of ε-aminocaproic acid on activity of human plasmin and recombinant truncated plasmin in pig vitreous at 37° C.

### DETAILED DESCRIPTION OF THE INVENTION

As used herein, the terms “autodegradable enzyme” and “autolyzable enzyme” are used interchangeably and mean an enzyme that is capable of breaking down, digesting, degrading, or hydrolyzing its own molecule due to its enzymatic or catalytic activity. The term “physiological pH” means pH of about 7.2-7.6.

In general, the present invention provides a composition comprising an enzyme that has been preserved at a pH less than about 5 and is subsequently reconstituted in a formulation comprising a material selected from the group consisting of tranexamic acid, ε-aminocaproic acid, analogs of L-lysine other than tranexamic acid and ε-aminocaproic acid, combinations thereof, and mixtures thereof; wherein said enzyme is autodegradable at a pH greater than about 5. The term “combination” encompasses, but is not limited to, two or more molecules attached, attracted, held, or adhered together by bonds (hydrogen bonding, ionic bonding, physical (such as by van der Waals force) or chemical adsorption, covalent bonding, or organometallic interaction), two interpenetrating molecules, or a complex comprising two or more molecules by, e.g., bonding or conformational interaction.

In one embodiment, the material is tranexamic acid.

In another embodiment, the material is a combination or mixture of tranexamic acid and ε-aminocaproic acid.

In still another embodiment, the material is an analog of L-lysine other than tranexamic acid and ε-aminocaproic acid. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In still another embodiment, the material is a mixture of tranexamic acid and an analog of L-lysine other than tranexamic acid and ε-aminocaproic acid.

In still another aspect, the formulation used in the composition further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), streptokinase, tPA, uPA, combinations thereof, and mixtures thereof; and Group 3 consists of non-ionic surfactants, glycerin, D-sorbitol, combinations thereof, and mixtures thereof. Betaine is also known as (carboxymethyl)trimethylammonium inner salt or oxyneurine. Sarcosine is also known as N-methylglycine.

In a further aspect, the formulation comprises a mixture of: (1) tranexamic acid, ε-aminocaproic acid, or an analog of L-lysine other than tranexamic acid and ε-aminocaproic acid; and (2) a compound of either Group 1, Group 2, or Group 3.

In yet another aspect, the formulation has a pH corresponding approximately to a pH at which said enzyme has the highest activity in a preselected reaction or use.

Said materials and said compounds of Groups 1, 2, and 3 are herein sometimes referred to collectively as “additives.”

In one embodiment, the compound is selected from Group 1. In another embodiment, the compound is selected from Group 2. In still another embodiment, the compound is selected from Group 3. In yet another embodiment, the compound is selected from the group of non-ionic surfactants.

In some embodiments, Group 1 consists of L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, streptokinase, tPA, uPA, combinations thereof, and mixtures thereof; and Group 3 consists of non-ionic surfactants, glycerin, combinations thereof, and mixtures thereof.

In one aspect, the pH of the formulation is in the range from about 6.5 to about 11. Alternatively, the pH of the formulation is in the range from about 6.5 to about 9, or from about 6.5 to about 8. In another aspect, the formulation comprises a buffer having a pH in one of said pH ranges.

In another aspect, the pH of the formulation changes by less than about 1 pH unit (or alternatively, less than about 0.5, or about 0.2, or about 0.1 pH unit) when the preserved enzyme is added into the formulation.

In yet another embodiment, a composition of the present invention comprises an enzyme that has been preserved at a pH less than about 5 and is subsequently reconstituted in a formulation comprising tranexamic acid and a compound selected from Group 2; wherein said enzyme is autodegradable at a pH greater than about 5. The formulation can have a pH corresponding approximately to a pH at which said enzyme has the highest activity in a preselected reaction or use.

In a further embodiment, a composition of the present invention comprises an enzyme that has been preserved, at a pH less than about 5 and is subsequently reconstituted in a formulation comprising: (a) ε-aminocaproic acid; and (b) a compound selected from Group 2; wherein said enzyme is autodegradable at a pH greater than about 5. In another embodiment, the enzyme has been preserved at a pH less than about 4 (or alternatively, at a pH in the range from about 2.5 to bout 4, or from about 2.5 to about 3.5).

In yet another embodiment, the formulation has a pH corresponding approximately to a pH at which said enzyme has the highest activity in a preselected reaction or use.

In one aspect, the concentration of tranexamic acid, ε-aminocaproic acid, or another analog of L-lysine other than tranexamic acid and ε-aminocaproic acid in any formulation disclosed herein is in a range from about 1 μM to about 500 mM (or alternatively, from about 10 μM to about 200 mM, or from about 50 μM to about 100 mM, or from about 50 μM to about 20 mM).

In another aspect, the concentration of the compound in any formulation disclosed herein is in a range from about 0.001 to about 5 weight percent (or alternatively, from about 0.01 to about 4 weight percent, or from about 0.01 to about 2 weight percent).

In one embodiment, a composition of the present invention comprises an enzyme that has been preserved at a pH less than about 5 and is subsequently reconstituted in a formulation comprising: (a) a mixture of tranexamic acid and ε-aminocaproic acid; and (b) a compound selected from Group 2; wherein said enzyme is autodegradable at a pH greater than about 5.

In one aspect, the concentration of each of tranexamic acid and ε-aminocaproic acid in the formulation is in a range from about 1 μM to about 500 mM (or alternatively, from about 10 μM to about 200 mM, or from about 50 μM to about 100 mM, or from about 50 μM to about 20 mM).

In one embodiment, the compound is HSA. In another embodiment, the compound is gelatin. In still another embodiment, the compound is a mixture of HSA and gelatin.

Although applicants do not wish to be bound by any particular theory, it is believed that an additive binds reversibly to certain active regions of an enzyme molecule, thereby preventing it from catalyzing the break down of itself or other molecules of the same enzyme. Different additives may bind to different regions of the enzyme molecule and provide a synergistic inhibiting effect.

In another aspect, the present invention provides a method for producing an active enzyme after prolonged storage, the method comprising: (a) storing said enzyme at a pH less than about 5; and (b) adding said enzyme to a formulation that comprises a material selected from the group consisting of tranexamic acid, ε-aminocaproic acid, analogs of L-lysine other than tranexamic acid and ε-aminocaproic acid, combinations thereof, and mixtures thereof; wherein said enzyme is autodegradable at a pH greater than about 5; to produce a formulated enzyme substantially immediately before using the enzyme or carrying out the reaction, wherein said enzyme is autodegradable at pH greater than about 5. Non-limiting examples of analogs of L-lysine are disclosed above.

In still another aspect, the formulation used in the foregoing method further comprises a compound selected from Group 1, Group 2, and Group 3; wherein Group 1 consists of L-lysine, L-arginine, L-ornithine, γ-aminobutyric acid, glycylglycine, combinations thereof, and mixtures thereof; Group 2 consists of gelatin, HSA, combinations thereof, and mixtures thereof; and Group 3 consists of non-ionic surfactants, glycerin, combinations thereof, and mixtures thereof.

In one embodiment, the compound is selected from the group of non-ionic surfactants. In another embodiment, the non-ionic surfactants are selected from the group consisting of polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F68, Pluronic® F127, or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34 ed., pp 1411-1416 (Martindale, “The complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006); the contents of these sections are incorporated herein by reference.

In yet another aspect, the pH of the formulation changes by less than about 1 pH unit (or alternatively, less than about 0.5, or less than about 0.2, or less than about 0.1 pH unit) when the preserved enzyme is added into the formulation. The relative amounts of the enzyme, the material, the compound (when present), and other constituents of the formulation (when present) are thus chosen based on the desired maximum change in the pH of the solution, the enzyme, the type of material, the type of the compound (when present), and the types of other constituents (when present) without difficulty.

In one aspect, the concentration of tranexamic acid or ε-aminocaproic acid in any formulation used in any method disclosed herein is in a range from about 1 μM to about 500 mM (or alternatively, from about 10 μM to about 200 mM, or from about 50 μM to about 100 mM, or from about 50 μM to about 20 mM).

In another aspect, the concentration of the compound in any formulation used in any method disclosed herein is in a range from about 0.001 to about 5 weight percent (or alternatively, from about 0.01 to about 4 weight percent, or from about 0.01 to about 2 weight percent).

In another aspect, the step of storing of said enzyme is effected at a pH less than about 4. Alternatively, said pH is less than 3.5 or in the range from about 2.5 to about 4, or from about 2.5 to about 3.5, or from about 3 to about 3.5.

The present invention is useful in producing an active enzyme after prolonged storage after its manufacture. Such an enzyme is reconstituted in a composition and is available for use, such as a therapeutic or diagnostic use, after a prolonged storage. In particular, the present invention provides a solution to the problem of decay of activity of autodegradable enzymes upon storage, which have not been adopted for wide use because of such autodegradation or autolysis.

In one aspect, the enzyme is a proteolytic enzyme. In another aspect, the enzyme is selected from the group consisting of serine proteinases, cysteine proteinases, aspartyl proteinases, metalloproteinases, derivatives thereof, combinations thereof, and mixtures thereof. In still another aspect, the enzyme is selected from the group consisting of serine proteinases. Non-limiting examples of such serine proteinases include plasmin, trypsin, chymotrypsin, elastase, carboxypeptidase, derivatives thereof, combinations thereof, and mixtures thereof.

As used herein, “derivatives” of an enzyme encompass variants of the enzyme that still substantially retain the basic enzymatic function of the enzyme. Such variants can be modified forms of the enzyme, such as for example a truncated form wherein one or more amino acid residues or segments of the enzyme molecule are deleted. Such variants also can be a form of the enzyme wherein one or more amino acid residues are substituted, such as by conservative substitutions, or wherein one or more amino acid residues are added to the polypeptide. In one aspect, the enzyme is plasmin or a derivative thereof. As used herein, a derivative of plasmin encompasses a polypeptide that is a fragment or portion thereof that can comprise the enzymatic or catalytic domain or region of plasmin. A derivative of plasmin can further comprise a kringle domain or region of the plasmin molecule. A kringle domain of plasmin is characterized by a triple-loop conformation and comprises about 75-85 amino acid residues with three disulfide bridges. Within the scope of derivatives of plasmin is microplasmin, which comprises the serine proteinase enzymatic domain of plasmin and a short polypeptide sequence (e.g., comprising about 25-40 amino acid residues) between the enzymatic domain and where it would normally be connected to the kringle-5 domain of plasmin.

In another aspect, a derivative of plasmin can be a miniplasmin, which comprises the kringle-5 domain and the enzymatic domain of plasmin. Enzymatically active microplasmin and miniplasmin are obtained from microplasminogen and miniplasminogen precursors by cleavage of the peptide bond at Arg⁵⁶¹-Val⁵⁶², wherein the amino acid residue numbers correspond to those of human Glu-plasminogen, which has 791 amino acid residues. Microplasminogen and miniplasminogen are disclosed in U.S. Patent Application Publications 2004/0071676 A1 and 2005/0124036 A1, which are incorporated herein by reference in their entirety.

In another aspect, a derivative of plasmin can comprise one or more kringle domains (i.e., one or more kringle-1, -2, -3, -4, and -5) attached in any order to the enzymatic domain.

In still another aspect, a derivative of plasmin can be a material known as angiostatin, which comprises only one or more kringle domains of plasmin, without its enzymatic domain, such as 3 to 5 contiguous kringle domains.

Plasmin can be produced by activation of plasminogen precursor, which may be obtained from plasma. For example, a method of producing high-purity plasmin is disclosed in U.S. Patent Application Publication 2004/0171103 A1, which is incorporated herein by reference in its entirety. The starting material, plasminogen, can be extracted from Cohn Fraction II+III paste by affinity chromatography on Lys-SEPHAROSE™ as described by D. G. Deutsch and E. T. Mertz, “Plasminogen: purification from human plasma by affinity chromatography,” Science 170(962):1095-6 (1970). (SEPHAROSE™ is a trade name of Pharmacia, Inc., New Jersey.)

Following the extraction of plasminogen from the Cohn Fraction II+III paste, lipid and protein impurities and Transmissible Spongiform Encephalopathies (“TSE”) contaminants are reduced by precipitation with the addition of polyethylene glycol (“PEG”), in a range of about 1 to about 10 percent weight/volume or the addition of about 80 to about 120 g/l ammonium sulfate. The PEG or ammonium sulfate precipitate is removed by depth filtration and the resulting solution placed on a lysine affinity resin column. The phrase “lysine affinity resin” is used generally for affinity resins containing lysine or its derivatives or ε-aminocaproic acids as the ligand. The column can be eluted with a solution having a low pH of approximately 1 to 4.

The protein obtained after elution from the affinity column is generally at least 80 percent plasminogen. The purified plasminogen is then stored at low pH in the presence of simple buffers such as glycine and lysine or w-amino acids.

Plasminogen in solution is then activated to plasmin by the addition of a plasminogen activator, which may be accomplished in a number of ways including but not limited to streptokinase, urokinase, tissue plasminogen activator (“tPA”), or the use of urokinase immobilized on resin and use of streptokinase immobilized on resin. In one embodiment, the plasminogen activator is soluble streptokinase. The addition of stabilizers or excipients such as glycerol, ω-amino acids such as lysine, polylysine, arginine, ε-aminocaproic acid and tranexamic acid, and salt can enhance the yield of plasmin.

Plasmin can be purified from unactivated plasminogen by affinity chromatography on resin with benzamidine as the ligand and eluted preferably with a low pH solution (e.g., pH<4, or alternatively pH between about 2.5 and about 4). This step can remove essentially all degraded plasmin as well as the majority of the streptokinase.

As a polishing step for the removal of remaining streptokinase, hydrophobic interaction chromatography (“HIC”) at low pH is performed (e.g., pH<4). Following the HIC step, plasmin is formulated as a sterile protein solution by ultrafiltration and diafiltration and 0.22-μm filtration.

The eluted plasmin from such polishing step can be buffered with a low pH (e.g., pH<4), low buffering capacity agent. The low pH, low buffering capacity agent typically comprises a buffer of either an amino acid, a derivative of at least one amino acid, an oligopeptide which includes at least one amino acid, or a combination thereof. In addition, the low pH, low buffering capacity agent can comprise a buffer selected from acetic acid, citric acid, hydrochloric acid, carboxylic acid, lactic acid, malic acid, tartaric acid, benzoic acid, serine, threonine, methionine, glutamine, alanine, glycine, isoleucine, valine, alanine, aspartic acid, derivatives, and combinations thereof. The concentration of plasmin in the buffered solution can range from about 0.01 mg/ml to about 50 mg/ml of the total solution. The concentration of the buffer can range from about 1 nM to about 50 mM. Of course, these ranges may be broadened or narrowed depending upon the buffer chosen, or upon the addition of other ingredients such as additives or stabilizing agents. The amount of buffer added is typically that which will give the reversibly inactive acidified plasmin solution a pH between about 2.5 to about 4, or between about 3 and about 3.5.

It may be advantageous to add a stabilizing or bulking agent to the reversibly inactive acidified plasmin solution obtained as disclosed above. Non-limiting examples of such stabilizing or bulking agents are a polyhydric alcohols, pharmaceutically acceptable carbohydrates, salts, glucosamine, thiamine, niacinamide, and combinations thereof. The stabilizing salts can be selected from the group consisting of sodium chloride, potassium chloride, magnesium chloride, calcium chloride, and combinations thereof. Sugars or sugar alcohols may also be added, such as glucose, maltose, mannitol, sorbitol, sucrose, lactose, trehalose, and combinations thereof. Other carbohydrates that may be used are polysaccharides, such as dextrin, dextran, glycogen, starches, carboxymethylcellulose, derivatives thereof, and combinations thereof. Concentrations of a carbohydrate added to add bulk to the reversibly inactive acidified plasmin solution can be in a range from about 0.2 percent weight/volume (“% w/v”) to about 20% w/v. Concentrations for a salt, glucosamine, thiamine, niacinamide, and their combinations can range from about 0.01 M to about 1 M.

Plasmin formulated according to the method disclosed above in buffered acidified water has been found to be very stable. It can be kept in this form for months without substantial loss of activity or the appearance of degradation products of a proteolytic or acidic nature. At 4° C., such plasmin is stable for at least nine months. Even at room temperature, such plasmin is stable for at least two months.

Inactive acidified plasmin compositions including a bulking agent, such as a carbohydrate, can be optionally lyophilized at a temperature in a range, for example, from about 0° C. to about −50° C., or preferably from about 0° C. to about −20° C., to produce a powder for long-term storage.

In another aspect, plasmin or variants thereof can be produced by recombinant technology, and a method of the present invention is applied to such plasmin and variants thereof. For example, the production of recombinant microplasminogen (which can be activated to microplasmin by cleavage of the peptide bond at Arg⁵⁶¹-Val⁵⁶² using one of the plasminogen activators disclosed above) in the Pichia pastoris yeast system is disclosed in U.S. Patent Application Publication 2004/0071676 A1, which is incorporated herein by reference. Plasminogen and miniplasminogen (which also can be activated to miniplasmin by cleavage of the peptide bond at Arg⁵⁶¹-Val⁵⁶² using one of the plasminogen activators disclosed above) in the Pichia pastoris yeast system is disclosed in U.S. Patent Application Publication 2005/0124036 A1, which is incorporated herein by reference.

Recombinant plasmin or variants thereof are acidified and stored at pH less than about 5 (or alternatively less than about 4, or between about 2.5 and about 3.5). The acidified enzyme is reconstituted by adding said enzyme to a formulation having pH corresponding approximately to a pH at which said enzyme has the highest activity in a preselected reaction or use and containing one or more additives disclosed above, to produce a formulated enzyme substantially immediately before using the enzyme or carrying out the reaction. In one embodiment, the formulation has a buffering capacity such that the pH of the formulation changes by less than about 1 pH unit upon adding said enzyme. In another embodiment, said formulation has a buffering capacity such that the pH of the formulation changes by less than about 0.5 (or alternatively, less than about 0.2, or less than about 0.1) pH unit upon adding said enzyme.

In one aspect, the formulation has a pH of about 7. Alternatively, the formulation has a pH in a range from about 7 to about 7.5.

In another aspect, the formulation has a pH of about 7.4.

In still another aspect, the formulation comprises a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(Cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In a further aspect, the present invention provides a method for prolonging an activity of plasmin or derivatives thereof in a posterior chamber of an eye, the method comprising: (a) providing said plasmin or derivatives thereof that have been preserved at a pH less than about 5; and (b) adding said plasmin or derivatives thereof to a formulation that comprises a material selected from the group consisting of tranexamic acid, ε-aminocaproic acid, analogs of L-lysine other than tranexamic acid and ε-aminocaproic acid, combinations thereof, and mixtures thereof, to produce a formulated plasmin or derivatives thereof; before administering said formulated plasmin or derivatives thereof into the posterior chamber of the eye, thereby prolonging the activity of plasmin or derivatives thereof in said posterior chamber of the eye; wherein the post-administering activity is higher than the activity of plasmin or derivatives thereof in a formulation without such material administered in said posterior chamber of the eye. The activity of plasmin or derivatives thereof reconstituted in such a formulation when administered into the posterior chamber will decay more slowly than that of plasmin or derivatives thereof reconstituted in a formulation without such material, such as saline solution.

In one embodiment, the formulation has a pH in the range from about 6.5 to about 11 (or alternatively, from about 6.5 to about 9, or from about 6.5 to about 8).

In another embodiment, the formulation has a buffering capacity such that a pH of buffered solution of said plasmin or derivatives thereof remains within about 1 pH unit (alternatively, within about 0.5, or 0.2, or 0.1 pH unit) upon adding said plasmin or derivatives thereof.

In still another aspect, the formulation further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, HSA, streptokinase, combinations thereof, and mixtures thereof; and Group 3 consists of non-ionic surfactants, glycerin, D-sorbitol, combinations thereof, and mixtures thereof. In yet another aspect, Group 1 consists of L-lysine, L-arginine, L-ornithine (or its pharmaceutically acceptable salts), γ-aminobutyric acid, glycylglycine, combinations thereof, and mixtures thereof; Group 2 consists of gelatin, HSA, combinations thereof, and mixtures thereof; and Group 3 consists of non-ionic surfactants.

In still another aspect, the present invention provides a kit for making an active enzyme or derivatives thereof. The kit comprises: (a) the enzyme or derivatives thereof that have been preserved at a pH less than about 5; and (b) a formulation that comprises a material selected from the group consisting of tranexamic acid, ε-aminocaproic acid, analogs of L-lysine other than tranexamic acid and ε-aminocaproic acid, combinations thereof, and mixtures thereof, provided in a separate container or package. In one embodiment, said formulation further comprises a compound selected from Group 1, Group 2, and Group 3, wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, HSA, streptokinase, tPA, uPA, combinations thereof, and mixtures thereof; and Group 3 consists of non-ionic surfactants, glycerin, D-sorbitol, combinations thereof, and mixtures thereof. In another embodiment, said formulation has a buffering capacity such that the pH of the formulation remains within about 1 pH unit upon adding said plasmin or derivatives thereof into said formulation.

In still another aspect, the present invention provides a method for inducing PVD in an eye, the method comprising: (a) providing said plasmin or derivatives thereof that have been preserved at a pH less than about 5; and (b) adding said plasmin or derivatives thereof to a formulation that comprises a material selected from the group consisting of tranexamic acid, ε-aminocaproic acid, analogs of L-lysine other than tranexamic acid and ε-aminocaproic acid, combinations thereof, and mixtures thereof, to produce a formulated plasmin or derivatives thereof; before administering said formulated plasmin or derivatives thereof into the posterior chamber of the eye, thereby inducing PVD in said eye. In one embodiment, said formulation has a buffering capacity such that a pH of said formulated plasmin or derivatives thereof remains within about 1 (or alternatively, within about 0.5, or about 0.2, or about 0.1) pH unit upon adding said plasmin or derivatives thereof.

In still another aspect, the formulation used in the foregoing method further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, human serum albumin (“HSA”), streptokinase, tPA, uPA, combinations thereof, and mixtures thereof; and Group 3 consists of non-ionic surfactants, glycerin, D-sorbitol, combinations thereof, and mixtures thereof.

Non-limiting amounts or concentrations of the various materials or compounds disclosed above are also applicable to the various methods of the present invention disclosed herein.

In still another aspect, the method of the present invention has an advantage of substantially preventing precipitation of said plasmin or derivatives thereof in said posterior chamber of the eye upon administering said plasmin or derivatives thereof.

In a further aspect, the present invention provides a method of inducing PVD in an eye, the method comprising administering a formulation of plasmin or derivatives thereof into a posterior chamber of an eye of a patient in need of having PVD; wherein said plasmin or derivatives thereof have been preserved at a pH less than about 5; and said formulation further comprises a material selected from the group consisting of tranexamic acid, ε-aminocaproic acid, analogs of L-lysine other than tranexamic acid and ε-aminocaproic acid, combinations thereof, and mixtures thereof, thereby inducing PVD in said eye.

In one aspect, the patient may be one who has symptoms of the beginning of a pathological PVD and the method induces a controlled PVD. Such a controlled PVD can arrest or prevent damage to the retina, which would occur if the pathological uncontrolled PVD is allowed to continue.

In one embodiment, said formulation further comprises a compound selected from Group 1, Group 2, and Group 3; wherein Group 1 consists of L-lysine, L-arginine, L-ornithine (or its pharmaceutically acceptable salts; e.g., L-ornithine hydrochloride), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, HSA, streptokinase, tPA, uPA, combinations thereof, and mixtures thereof; and Group 3 consists of non-ionic surfactants, glycerin, D-sorbitol, combinations thereof, and mixtures thereof. In another embodiment, said formulation further comprises a compound selected from Group 1, Group 2, and Group 3; wherein Group 1 consists of L-lysine, L-arginine, L-ornithine, γ-aminobutyric acid, glycylglycine, combinations thereof, and mixtures thereof; Group 2 consists of gelatin, HSA, combinations thereof, and mixtures thereof; and Group 3 consists of non-ionic surfactants, glycerin, combinations thereof, and mixtures thereof.

In another embodiment, said formulation is made by adding plasmin or derivatives thereof to a solution containing an additive selected from said materials and said compounds, substantially immediately before use.

In another embodiment, said formulation is administered in an amount containing a therapeutically effective amount of plasmin or derivatives thereof to induce said PVD.

Method of injecting plasmin or derivatives thereof into eye for PVD is now described.

Plasmin or derivatives thereof, which is reconstituted substantially immediately before administering into a patient with a formulation comprising one or more additives as disclosed above, can be injected intravitreally, for example through the pars plana of the ciliary body, to induce controlled PVD using a fine-gauge needle, such as 25-30 gauge. Administration of plasmin or derivatives thereof can be used to prevent, treat, or ameliorate the potentially blinding complications of an ocular condition, such as diabetic retinopathy, retinal detachment, macular edema, macular hole, and retinal tears. Typically, an amount from about 25 μl to about 100 μl of a composition comprising about 1-5 IU of plasmin or derivatives thereof per 50 μl of formulation is administered into the vitreous. Alternatively, a composition can comprise about 0.01-50 mg/ml (or about 0.1-10 mg/ml, or about 0.2-5 mg/ml, or about 0.5-4 mg/ml) of plasmin or derivatives thereof. Such administration of plasmin or derivatives thereof may be periodically repeated upon assessment of the treatment results and recommendation by a skilled medical practitioner. For example, it may be found appropriate to repeat such administration every 4-6 weeks, or every 3 months, or every 6 months, depending on the condition.

In yet another aspect, the present invention provides a method for preventing or reducing precipitation of an enzyme administered into a region of a patient, the method comprising: (a) providing the enzyme at a pH of less than about 5; (b) adding said enzyme to a formulation that comprises a material selected from the group consisting of tranexamic acid, ε-aminocaproic acid, analogs of L-lysine other than tranexamic acid and ε-aminocaproic acid, combinations thereof, and mixtures thereof; to produce a formulated enzyme before administering said formulated enzyme into said region of the patient. In one embodiment, the formulation has a pH in the range from about 6.5 to about 11 (or alternatively, from about 6.5 to about 9, or from about 6.5 to about 8). In another embodiment, upon adding the enzyme to the formulation, the pH of the formulation remains within about 1 pH unit (or alternatively, within about 0.5, or about 0.2, or about 0.1 pH unit) of the original formulation pH. In still another embodiment, the formulation comprises a buffer. In a further embodiment of the present invention, said region of the patient is a vitreous of an eye or a circulatory system.

In one aspect, the formulation has a pH of about 7. Alternatively, the formulation has a pH in a range from about 7 to about 7.5.

In another aspect, the formulation has a pH of about 7.4.

In still another aspect, the formulation comprises a phosphate buffer or a Tris-HCl buffer. In yet another aspect, the formulation comprises 10× phosphate buffered saline (“PBS”) or 5×PBS solution.

### Example 1

### Plasmin Precipitation Study in Buffer Solutions

Sterile, purified, and unbuffered human plasmin (pH of 3.3±0.3) in a stable, lyophilized form and without any preservative was obtained from Talecris, Inc. (Research Triangle Park, N.C.). This acidified, lyophilized plasmin was reconstituted with 0.9% (by weight) NaCl solution to a concentration of 10 mg/ml. An aliquot of this reconstituted plasmin solution was transferred to a PBS buffer solution (pH of about 7.4) containing an additive selected from the group consisting of tranexamic acid (“TXA”), ε-aminocaproic acid (“ε-ACA”), γ-aminobutyric acid, 5-aminovaleric acid, 7-aminoheptanoic acid, glycylglycine, triglycine, L-ornithine hydrochloride, N-α-acetyl-L-arginine, L-arginine, betaine, sarcosine, D-sorbitol, glycerin, and gelatin. Combinations of ε-aminocaproic acid and gelatin or glycerin were also tested. The concentration of plasmin in the additive-containing buffer was 1 mg/ml. The solutions were observed for any precipitation within 2 hours following addition of plasmin, and the results are shown in Tables 1 and 2.

TABLE 1
Effects of Additives on Physical Appearance
of Plasmin in PBS, pH of about 7.4

| | | | | Minimum | |
|---|---|---|---|---|---|
| | | | | Concentration of | |
| | | | | Additive Required to | |
| | | | | Prevent Visually | |
| Test | | Concentration | | Observed Plasmin | |
| No. | Additive | Range Tested | | Precipitation | |
| 1 | tranexamic acid | 0.00005-0.005 | M | 0.5 | mM |
| 2 | ε-aminocaproic | 0.0001-0.1 | M | 1 | mM |
| | acid | | | | |
| 3 | γ-aminobutyric | 0.001-0.005 | M | 5 | mM |
| | acid | | | | |
| 4 | glycylglycine | 0.005-0.09 | M | 10 | mM |
| 5 | L-ornithine | 0.01-0.1 | m | 50 | mM |
| | hydrochloride | | | | |
| 6 | N-α-acetyl-L- | 0.005-0.05 | M | 50 | mM |
| | arginine | | | | |
| 7 | L-arginine | 0.005-1 | M | 50 | mM |
| 8 | glycerin | 2-20% | (v/v) | 10% | (by weight) |
| 9 | ε-aminocaproic | not available | | 0.1 | mM |
| | acid | | | | |
| | gelatin ⁽¹⁾ | not available | | 0.3% | (w/v) |
| 10 | ε-aminocaproic | not available | | 0.1 | mM |
| | acid | | | | |
| | glycerin | not available | | 6% | (v/v) |
| 11 | 5-aminovaleric | 0.0001-0.1 | M | 1 | mM |
| | acid | | | | |
| 12 | 7-aminoheptanoic | 0.001-0.005 | M | 5 | mM |
| | acid | | | | |
| 13 | triglycine | 0.002-0.1 | M | 10 | mM |
| 14 | betaine | 0.05-0.9 | M | 0.2 | M |
| 15 | sarcosine | 0.5-1 | M | 1 | M |
| 16 | D-sorbitol | 6-12% | (w/v) | 12% | (w/v) |

Note
⁽¹⁾ when gelatin (1.5%) was used as the additive, a slight precipitation was observed after incubating for 2 hours in PBS. For lower concentrations of gelatin (e.g., less than about 0.3%) precipitation was observed earlier than 2 hours.

TABLE 2
Effects of Other Additives on Physical Appearance
of Plasmin in PBS, pH of about 7.4

| Test | | Concentration Range Tested That Did Not | |
|---|---|---|---|
| No. | Additive | Prevent Precipitation at pH of About 7.4 | |
| 17 | HSA | 5% | (w/v) |
| 18 | Tween 20 ® | 0.1-1% | (v/v) |
| 19 | Tween 80 ® | 0.1-1% | v/v |
| 20 | propylene glycol | 2-20% | v/v |
| 21 | polyethylene glycol | 20% | v/v |
| | (“PEG”), MW = 400 | | |
| 22 | PEG, MW = 10,000 | 2-20% | w/v |
| 23 | Pluronic F68 ® | 0.02-2% | w/v |
| 24 | Pluronic F127 ® | 0.01-1% | w/v |
| 25 | Hp-β-cyclodextrin | 1 mM-50 mM | |
| 26 | N,N-dimethylglycine | 0.5-1 | M |
| 27 | sodium sulfate | 0.3-0.6 | M |
| 28 | oxalic acid | 50 | mM |
| 29 | adipic acid | 50 | mM |
| 30 | glycine | 0.1 | M |
| 31 | trehalose | 10% | |
| 32 | L-methionine | 0.2% | |

### Example 2

### Plasmin Precipitation Study in Rabbit Vitreous

In this study, the reconstituted acidified plasmin solution (10 mg/ml in 0.9% NaCl solution) formulated according to the procedure of Example 1, were used. An amount of this reconstituted acidified plasmin solution was added to a 0.9% NaCl solution containing one or more selected additives as shown in Table 2 below, to produce a plasmin formulation containing the additive or additives and a plasmin concentration of 1 mg/ml. An amount of 50 μl of each of the plasmin formulations was added to a 0.5 ml sample of homogenized young rabbit vitreous, which has a pH of about 8.5. The vitreous samples were observed for any precipitation within 2 hours following addition of plasmin, and the results are shown in Table 3.

TABLE 3
Effects of Additives on Plasmin Precipitation in Rabbit Vitreous

| Test | | | pH of the Final |
|---|---|---|---|
| No. | Additive | Precipitation | Formulation |
| 33 | 0.9% (by weight) | Yes | 3-4 |
| | NaCl (No additive) | | |
| 34 | 40 mM ε- | No | 6 |
| | aminocaproic acid | | |
| 35 | 40 mM ε- | No | 6 |
| | aminocaproic acid + | | |
| | 0.05% (by weight) | | |
| | polysorbate 80 | | |
| 36 | 40 mM ε- | No | 6.5 |
| | aminocaproic acid + | | |
| | 4% (by weight) HSA | | |
| 37 | 40 mM ε- | No | 6.3 |
| | aminocaproic acid + | | |
| | 4% (by weight) HSA + | | |
| | 1% gelatin | | |
| 38 | 40 mM tranexamic | No | 6 |
| | acid | | |
| 39 | 0.1 M N-α-acetyl-L- | No | 5.3 |
| | arginine | | |
| 40 | 50 mM glycylglycine | No | No data |
| 41 | 50 mM L-ornithine | No | No data |
| | hydrochloride | | |

### Example 3

### Plasmin Activity in Additive-Containing Buffers

In this study, the reconstituted acidified plasmin solution (10 mg/ml in 0.9% NaCl solution) and the buffered plasmin compositions containing selected additives, formulated according to the procedure of Example 1, were used. An amount of 50 μl of each of the buffered plasmin compositions was added to a 1.5 ml sample of PBS buffer (pH of about 7.4). The sample was stored at 37° C. Aliquots of the sample were collected at time 0, 1, 3, and 5 hours following addition of plasmin, and analyzed for plasmin activity by chromogenic assay using the plasmin substrate S-2251. S-2251 is a short peptide substrate for plasmin (H-D-Val-L-Leu-L-Lys-p-nitroaniline dihydrochloride, available from Chromogenix-Instrumentation Laboratory SpA, Milano, Italy). Plasmin hydrolyzes this substrate between the lysine residue and the p-nitroaniline moiety. The method determines the activity of plasmin based on the difference in absorbance (optical density) between the p-nitroaniline formed and the original substrate. The rate of p-nitroaniline formation; i.e., the increase in absorbance per second at wavelength of 405 nm, is proportional to the enzymatic activity of plasmin, and is conveniently measured with a photometer.

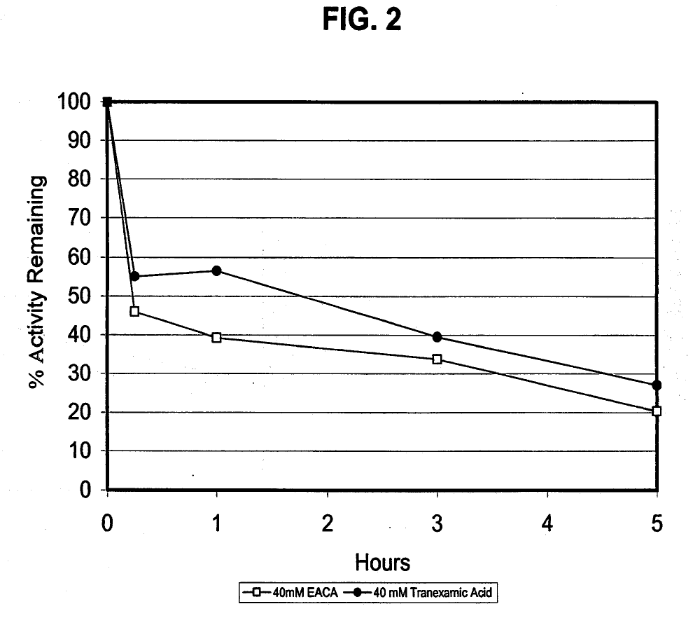

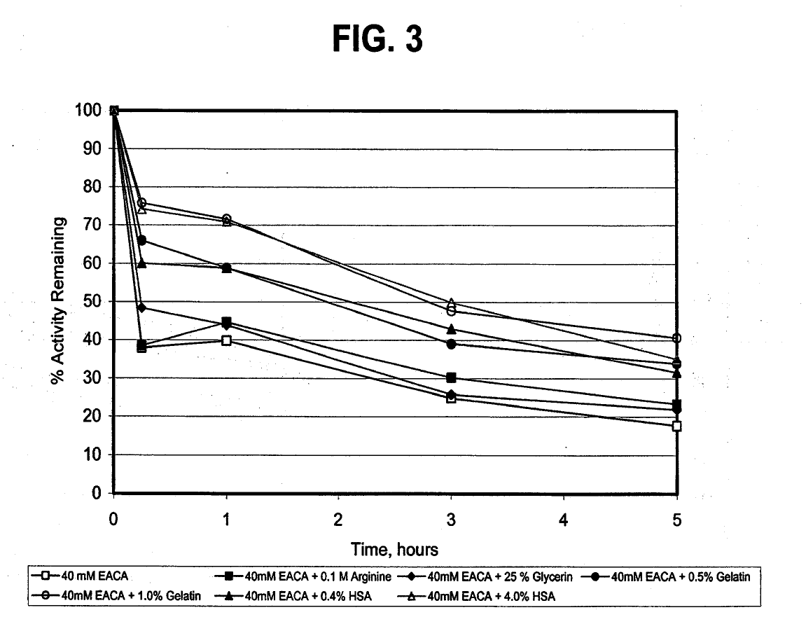

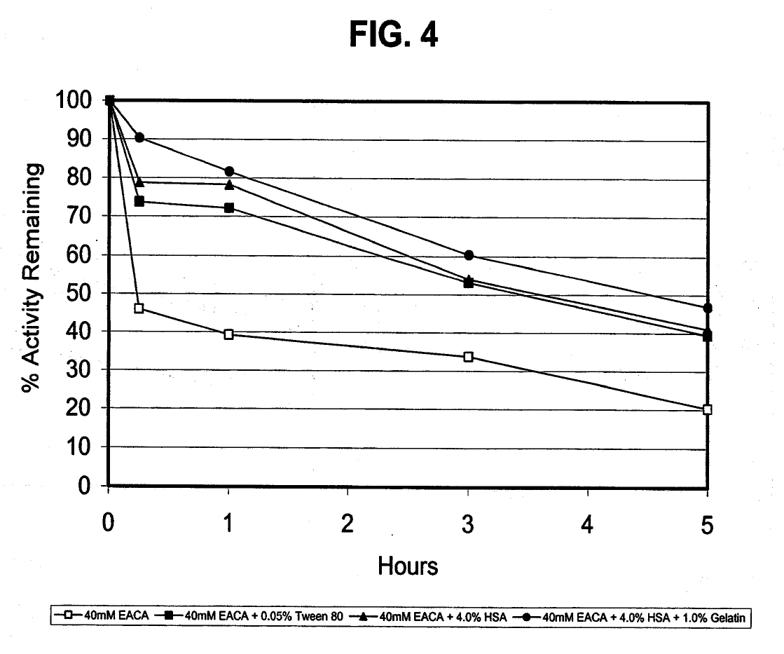

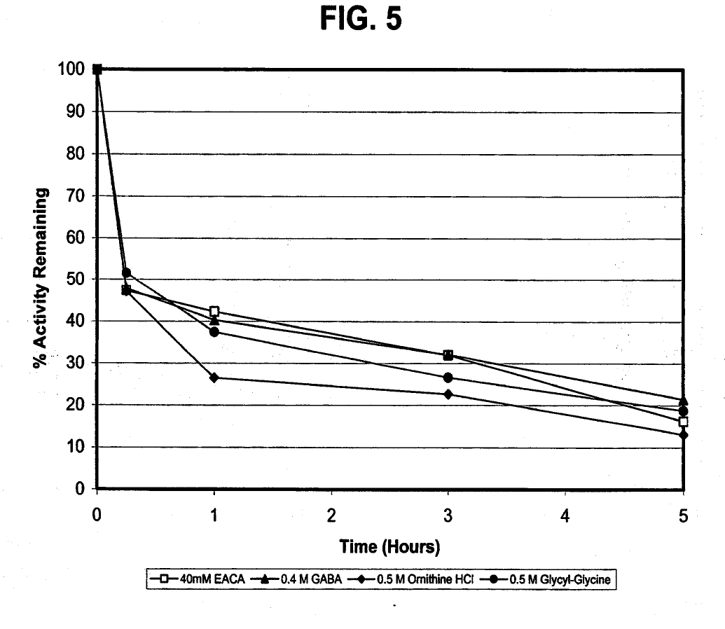

The following list shows various additives and additive combinations tested: 40 mM tranexamic acid, 40 mM ε-aminocaproic acid (“ε-ACA”), 40 mM ε-ACA+0.1 M arginine, 40 mM ε-ACA+25% (by weight) glycerine, 40 mM ε-ACA+0.5% (by weight) gelatin, 40 mM ε-ACA+1% (by weight) gelatin, 40 mM ε-ACA+0.4% (by weight) HSA, 40 mM ε-ACA+4% (by weight) HSA, 40 mM ε-ACA+4% (by weight) HSA+1% (by weight) gelatin, 40 mM ε-ACA+0.05% (by weight) polysorbate 80, 0.4 M γ-aminobutyric acid, 0.5 M L-ornithine hydrochloride, and 0.5 M glycylglycine. The results (as represented by activity relative to initial activity), shown in FIGS. 2-5, indicate that plasmin activity decays more slowly when it was reconstituted in compositions containing additives. The results of the wide range of additives tested indicate that many other combinations of the disclosed additives can provide a similar positive effect.

It should be noted that alternate chromogenic substrates for plasmin also may be used to determine its enzymatic activity, such as S-2390 (H-D-Val-L-Phe-L-Lys-p-nitroaniline dihydrochloride) or S-2403 (L-Pyroglutamyl-L-Phe-L-Lys-p-nitroaniline dihydrochloride); both are available from Chromogenix-Instrumentation Laboratory SpA, Milano, Italy.

### Example 4

### Another Study of Plasmin Activity in PBS Solutions Containing Selected Additives

Sterile, purified, and unbuffered human plasmin (pH of 3.3±0.3) in a stable, lyophilized form and without any preservative was obtained from Talecris, Inc. (Research Triangle Park, N.C.).

Three PBS buffer solutions were made according to the following formulations:

Formulation 1: L-lysine hydrochloride (5 mM), sodium phosphate monobasic (0.185% by weight), sodium phosphate dibasic (0.98% by weight), sodium chloride (0.4% by weight), and water (USP, q.s. to 100% by weight). This solution had a pH of about 7.4 and osmolarity of 308 mOsm/l.

Formulation 2: tranexamic acid (5 mM), sodium phosphate monobasic (0.185% by weight), sodium phosphate dibasic (0.98% by weight), sodium chloride (0.4% by weight), and water (USP, q.s. to 100% by weight). This solution had a pH of about 7.4 and osmolarity of 308 mOsm/l.

Formulation 3: tranexamic acid (100 μM), sodium phosphate monobasic (0.185% by weight), sodium phosphate dibasic (0.98% by weight), sodium chloride (0.4% by weight), and water (USP, q.s. to 100% by weight). This solution had a pH of about 7.4 and osmolarity of 308 mOsm/l.

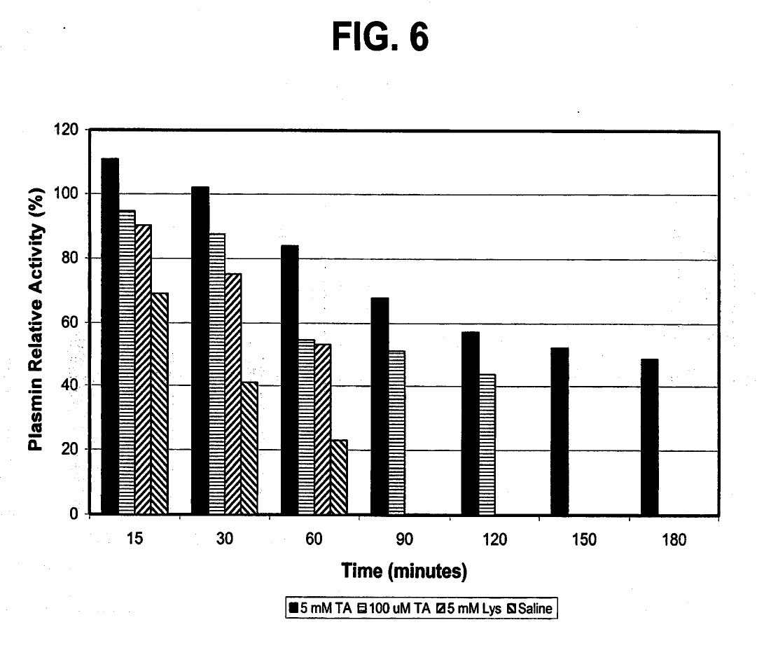

Plasmin (100 μg (equivalent to ˜4.7 IU)/50 μl) was mixed in a 1:4 ratio with each of the foregoing three PBS solutions. Normal saline was used instead of the PBS solution for control. An amount of 50 μl of each combination was added to 1 ml clear homogenized porcine vitreous, mixed thoroughly, and the mixture was incubated at 37° C. Plasmin activity was measured using the S-2251 chromogenic assay at time t=0, 15, 30, 60, 90, 120, 150, and 180 minutes. The activity of plasmin relative to initial activity is shown in FIG. 6. Plasmin reconstituted in PBS solutions containing an additive favorably retained its activity compared to the control sample (plasmin reconstituted in only saline solution).

### Example 5

### Human Plasmin Precipitation Study in Pig Vitreous

In this study, the reconstituted acidified human plasmin solution (10 mg/ml in 0.9% NaCl solution) and the plasmin compositions buffered at pH 3.5±0.3 containing selected additives in 0.9% NaCl solution were used. An amount of 50 μl of each of the formulated plasmin solution, 1 mg/ml, was added to a 1.5 ml sample of pig vitreous. The sample was stored at 37° C. The vitreous samples were observed for any precipitation within 2 hours following addition of plasmin, and the results are shown in Table 4.

TABLE 4
Effects of Additives on Physical Appearance
of Plasmin in Pig Vitreous

| Test | | |
|---|---|---|
| No. | Additive | Precipitation |
| 1 | 0.9% NaCl (No additive) | Yes |
| 2 | 5-40 mM ε-ACA + 0.05% Tween 80 ® ⁽¹⁾ | No |
| 3 | 2.5-10 mM ε-ACA + 0.01% Tween 80 ® | No |
| 4 | 1.25-20 mM TXA + 0.01% Tween 80 ® | No |
| 5 | 5 mM TXA + 0.05% Tween 80 ® | No |
| 6 | 5 mM TXA in Phosphate Buffered Saline, pH 7.4 | No |
| 7 | 0.1 M diglycine + 0.05% Tween 80 ® | No |
| 8 | 0.1 M triglycine + 0.05% Tween 80 ® | No |
| 9 | 5 mM 5-aminovaleric acid + 0.05% Tween 80 ® | No |

Note
⁽¹⁾ Tween 80 ® (also known as Polysorbate 80) is polyoxyethylene sorbitan monooleate surfactant.

### Example 6

### Stability of Human Plasmin in Pig Vitreous

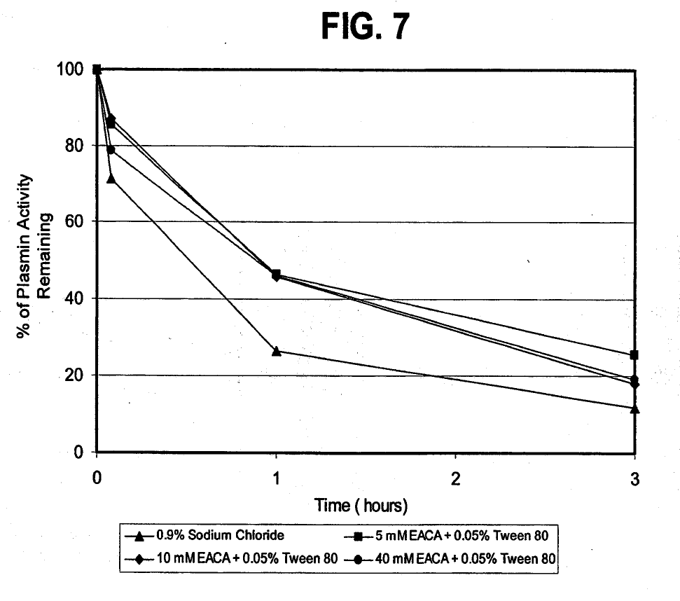

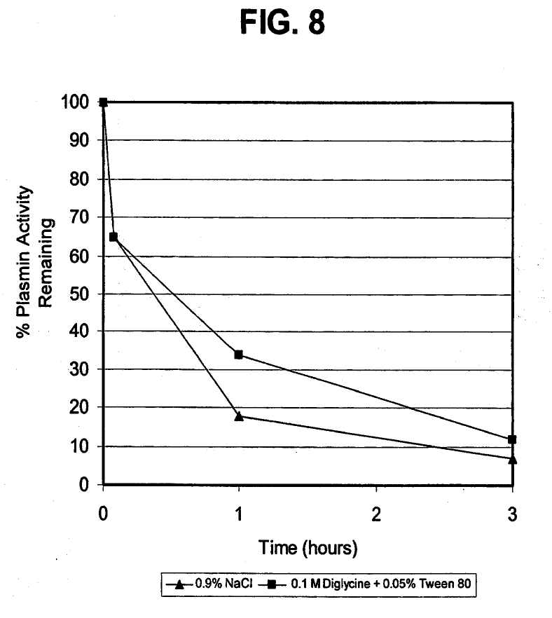

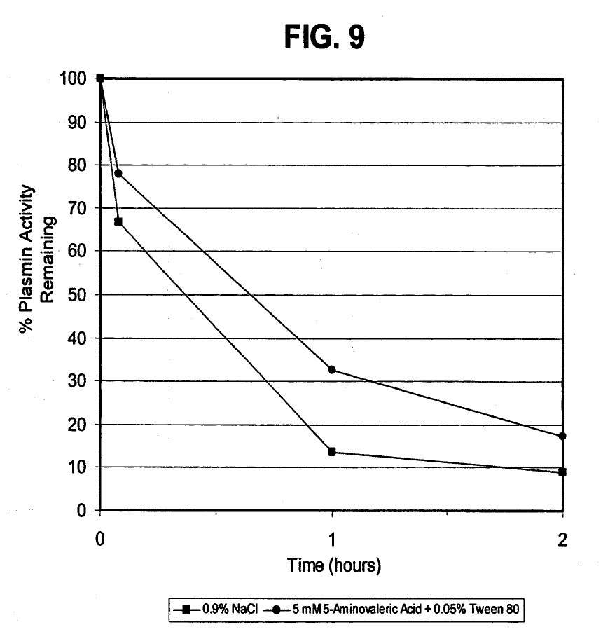

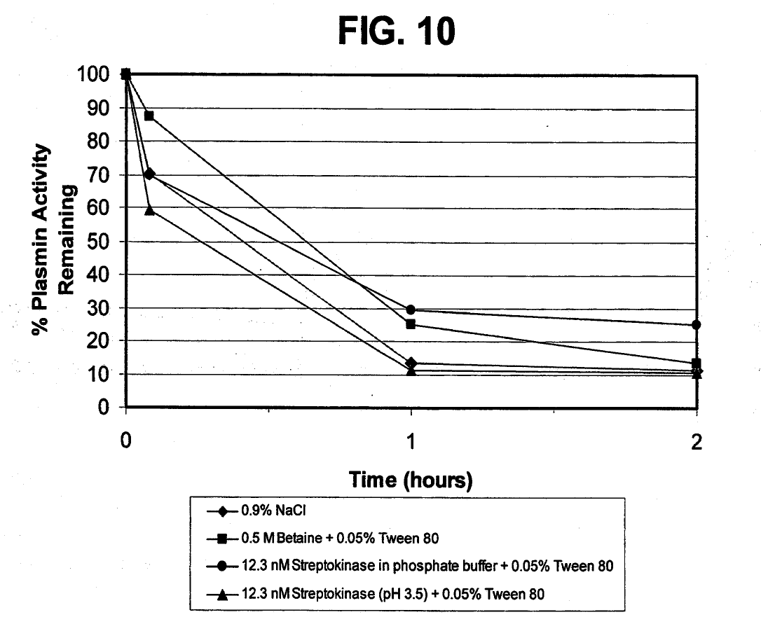

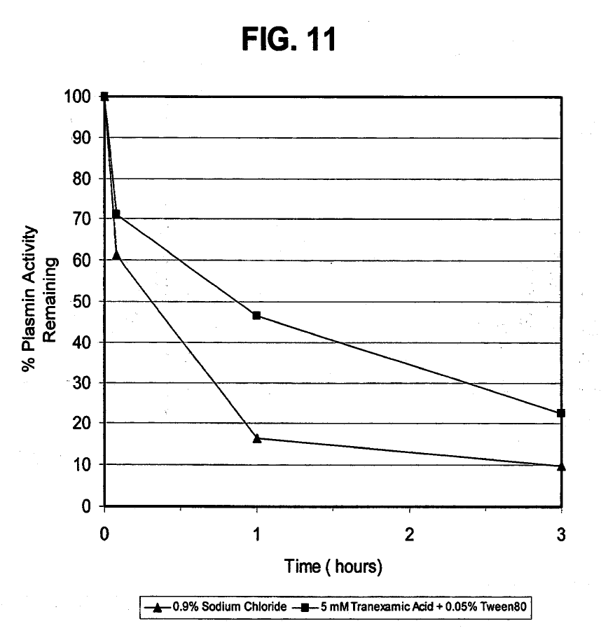

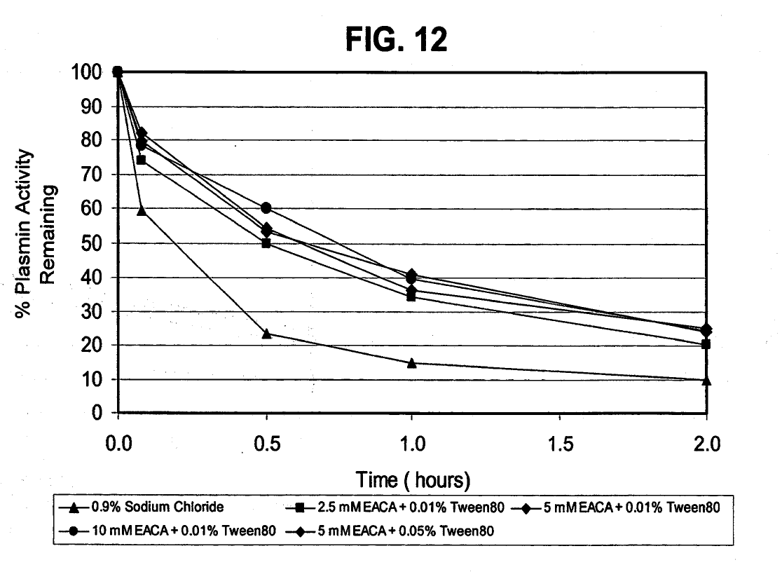

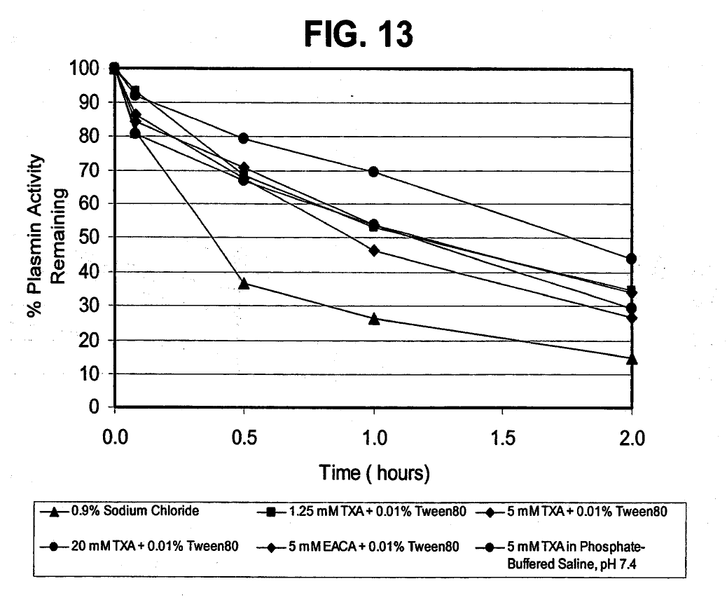

In this study, the reconstituted acidified plasmin solution (10 mg/ml in 0.9% NaCl solution) and the plasmin compositions buffered at pH 3.5±0.3 containing selected additives in 0.9% NaCl solution were used. An amount of 50 μl of each of the buffered plasmin formulation, 1 mg/ml, was added to a 1.5 ml sample of pig vitreous. The sample was stored at 37° C. Aliquots of the sample were collected at various time intervals following addition of plasmin, and analyzed for plasmin activity by chromogenic assay using the plasmin substrate S-2251. The following list shows various additive combinations tested: 5-40 mM ε-aminocaproic acid+0.05% Tween 80®, 0.1 M diglycine+0.05% Tween 80®, 5 mM 5-aminovaleric acid+0.05% Tween 80®, 0.5 M betaine+0.05% Tween 80®, 12.3 nM streptokinase (plasmin-to-streptokinase molar ratio of 1:1) in 3 mM phosphate buffer and 31 mM glutamate (purchased from Sigma-Aldrich)+0.05% Tween 80®, 5 mM tranexamic acid+0.05% Tween 80®, 2.5-10 mM ε-ACA+0.01% Tween 80®, 1.25-20 mM TXA+0.01% Tween 80®, 5 mM TXA in phosphate buffered saline, pH 7.4. The results (as represented by activity relative to initial activity), shown in FIGS. 7-13, indicate that plasmin activity decays more slowly when it was reconstituted in compositions containing additives. The results of the wide range of additives tested indicate that many other combinations of the disclosed additives can provide a similar positive effect.

### Example 7

### Stability of Recombinant Plasmin in Pig Vitreous

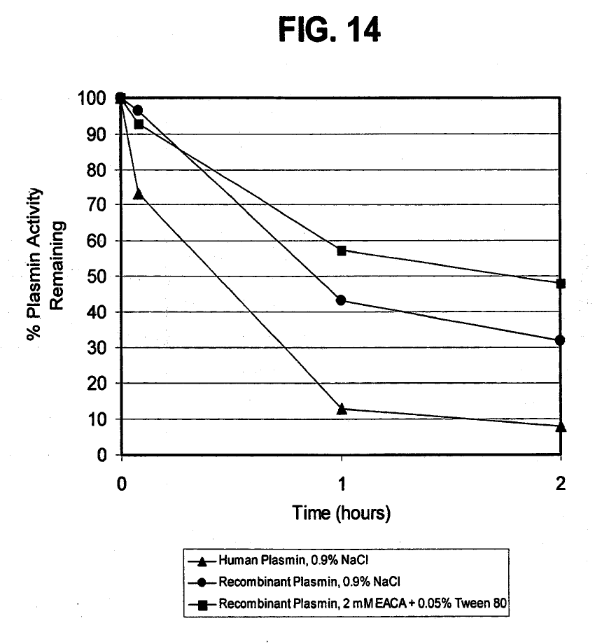

In this study, a truncated plasmin comprising the kringle-1 domain and the catalytic domain (M.W.=37198 Dalton), produced using recombinant technology, with a nominal concentration of 2.47 mg/ml (6.4 mg/ml by plasmin activity) in 0.9% NaCl solution, pH 3.5 and the plasmin compositions buffered at pH 3.5±0.3 containing selected additives in 0.9% NaCl solution were used. An amount of 50 μl of each of the buffered plasmin formulation, 1 mg/ml, was added to a 1.5 ml sample of pig vitreous. The sample was stored at 37° C. Aliquots of the sample were collected at 0, 1, and 2 hours following addition of plasmin, and analyzed for plasmin activity as described in Example 6. The results (as represented by activity relative to initial activity), shown in FIG. 14, indicate that plasmin activity decreases more slowly when it was reconstituted in compositions containing 2 mM ε-aminocaproic acid+0.05% Tween 80®.

### Example 8

### In Vivo Efficacy of Human Plasmin

In one in vivo efficacy study lasting 7 days in rabbits, using human-derived plasmin (obtained from Talecris, Inc.), the additive combination comprising 5 mM ε-ACA and 0.05% Tween 80® in 0.9% NaCl solution minimized or eliminated the haziness upon intravitreal injection at plasmin doses of 50-200 μg. In addition, this combination prolonged the plasmin activity and efficacy in vivo because a greater extent of PVD was obtained upon examination by SEM.

In another in vivo efficacy study lasting 14 days, using human-derived plasmin (obtained from Talecris, Inc.), the additive combination comprising 5 mM ε-ACA and 0.05% Tween 80® in 0.9% NaCl solution minimized or eliminated the haziness upon intravitreal injection at a plasmin dose of 200 μg. In addition, this combination prolonged the plasmin activity and efficacy in vivo because a greater extent of PVD was obtained upon examination by SEM.

In still another in vivo efficacy study lasting 14 days, using human-derived plasmin (obtained from Talecris, Inc.), the additive combinations comprising: (1) 0.5 mM tranexamic acid, 0.01% Tween 80®, and 2% trehalose in saline; and (2) 5-50 mM tranexamic acid, 0.01% Tween 80®, and 10% trehalose, in saline solution, minimized or eliminated the haziness upon intravitreal injection at a plasmin dose of 100 μg. In addition, these additive combinations prolonged the plasmin activity and efficacy in vivo because a greater extent of PVD was obtained upon examination by SEM.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

1-33. (canceled)

34. A method for inducing posterior vitreous detachment (PVD) in an eye, the method comprising:
(a) providing plasmin or derivatives thereof that have been preserved at a pH less than about 5; and
(b) adding said plasmin or derivatives thereof to a formulation that has a pH in a range from about 6.5 to about 11 and comprises a material selected from the group consisting of tranexamic acid, ε-aminocaproic acid, analogs of L-lysine other than tranexamic acid and 8-aminocaproic acid, combinations thereof, and mixtures thereof; to produce a formulated plasmin or derivatives thereof before administering said formulated plasmin or derivatives thereof into a posterior chamber of the eye, thereby inducing PVD in said eye.

35. The method of claim 34, wherein the formulation further comprises: (3) a compound selected from Group 1, Group 2, and Group 3; wherein Group 1 consists of L-lysine, L-arginine, L-ornithine (or its pharmaceutically acceptable salts), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, HSA, streptokinase, tPA, uPA, combinations thereof, and mixtures thereof; and Group 3 consists of non-ionic surfactants, glycerin, D-sorbitol, combinations thereof, and mixtures thereof.

36. The method of claim 35, wherein said formulation has a buffering capacity such that a pH of a formulated solution of said plasmin or derivatives thereof remains within about 1 pH unit upon adding said plasmin or derivatives thereof.

37. The method of claim 35, wherein precipitation of said plasmin or derivatives thereof in said posterior chamber of the eye is avoided upon administering said plasmin or derivatives thereof.

38. The method of claim 35, wherein said plasmin or derivatives thereof have been preserved at pH in a range from about 2.5 to about 4.

39. (canceled)

40. The method of claim 37, wherein the formulation further comprises a compound selected from Group 1, Group 2, and Group 3; wherein Group 1 consists of L-lysine, L-arginine, L-ornithine (or its pharmaceutically acceptable salts), γ-aminobutyric acid, 5-aminovaleric acid, 7-aminoheptanoic acid, glycylglycine, triglycine, N-α-acetyl-L-arginine, betaine, sarcosine, combinations thereof, and mixtures thereof; Group 2 consists of gelatin, HSA, streptokinase, tPA, uPA, combinations thereof, and mixtures thereof; and Group 3 consists of non-ionic surfactants, glycerin, D-sorbitol, combinations thereof, and mixtures thereof.

41. The method of claim 40, wherein upon adding the enzyme to the formulation, the pH of the formulation remains within about 1 pH unit of the pH of the formulation.

42. The method of claim 40, wherein said region of the patient is a vitreous of an eye.

43. The method of claim 40, wherein the pH of the enzyme of step (a) is in a range from about 2.5 to about 4.

44. The method of claim 40, wherein said enzyme is a proteolytic enzyme.

45. The method of claim 40, wherein said enzyme is selected from the group consisting of serine proteinases, cysteine proteinases, aspartyl proteinases, metalloproteinases, and combinations thereof.

46. The method of claim 40, wherein said enzyme is plasmin or plasmin derivatives.

47. The method of claim 40, wherein said region of a patient is a circulatory system of said patient.

48-52. (canceled)

53. A method of for inducing PVD in an eye, the method comprising administering a formulation of plasmin or derivatives thereof into a posterior chamber of an eye of a patient in need of having PVD; wherein said plasmin or derivatives thereof have been preserved at a pH less than about 5; and said formulation further comprises a material selected from the group consisting of tranexamic acid, ε-aminocaproic acid, analogs of L-lysine other than tranexamic acid and ε-aminocaproic acid, combinations thereof, and mixtures thereof, thereby inducing PVD in said eye.

54. The method of claim 53, wherein said formulation is made by adding plasmin or derivatives thereof to a solution containing a material selected from the group consisting of tranexamic acid, ε-aminocaproic acid, analogs of L-lysine other than tranexamic acid and ε-aminocaproic acid, combinations thereof, and mixtures thereof substantially immediately before said administering.

55. The method of claim 54, wherein said formulation further comprises a compound selected from Group 1, Group 2, and Group 3; wherein Group 1 consists of L-lysine, L-arginine, L-ornithine (or its pharmaceutically acceptable salts), γ-aminobutyric acid, 5-aminovaleric acid, 7-aminoheptanoic acid, glycylglycine, triglycine, N-σ-acetyl-L-arginine, betaine, sarcosine, combinations thereof, and mixtures thereof; Group 2 consists of gelatin, HSA, streptokinase, tPA, uPA, combinations thereof, and mixtures thereof; and Group 3 consists of non-ionic surfactants, glycerin, D-sorbitol, combinations thereof, and mixtures thereof.
